(19) United States
(12) Patent Application Publication
(10) Pub. No.: US 2014/0166799 A1
(43) Pub. Date: Jun. 19, 2014

(54) RECTRACTABLE OXYGEN SUPPLY-HOSE REEL AND MECHANISM HOUSING COVER FOR MEDICAL OXYGEN THERAPY DEVISES AND MEDICAL OXYGEN TREATMENT DEVISES.

(71) Applicant: Big E-Z Reel, LLC, Independence, LA (US)

(72) Inventors: James Jack Davis, JR., Independence, LA (US); Antonino F. Anzalone, Independence, LA (US); Vincent F. Falcone, JR., Independence, LA (US)

(73) Assignee: Big E-Z Reel, LLC

(21) Appl. No.: 13/925,521

(22) Filed: Jun. 24, 2013

Related U.S. Application Data
(60) Provisional application No. 61/739,184, filed on Dec. 19, 2012.

(51) Int. Cl. B65H 75/48; A61M 25/01

(52) U.S. Cl. CPC B65H 75/48; A61M 25/0113
USPC 242/382.1

(57) ABSTRACT

This invention modifies and redesigns a spring-loaded, retractable supply hose mechanisms used to pan out, retract, and store hoses used for gases (e.g., oxygen), treatments, or fluids, specifically this inventor's prior art, the Retractable Oxygen Supply Hose Mechanism, U.S. Pat. No. 6,065,490. This invention incorporates an “Adaptor Cap” or “Removable Cap” to prevent slippage of the Swivel connector, incorporates a “Removable Cap” into the upper disc or flange of the supply-hose reel and/or into the mechanism housing cover that acts as an “anti-rotational” device, and features bearing surfaces to prevent reel slippage. This internal ratchet system better prevents binding and kinking of the patient's supply, thereby facilitating the storage and the quick-controlled release of the desired amount of oxygen supply hose for patient's Medical Oxygen and other gases delivery, therapy, or treatments.

### CROSS REFERENCES TO RELATED APPLICATION

Not Applicable

### STATEMENT REGARDING FEDERALLY SPONSORED RESEARCH OR DEVELOPMENT

Not Applicable

### REFERENCE TO A MICROFILM APPENDIX

Not Applicable

### BACKGROUND OF THE INVENTION

This invention relates to the modifications and redesigning of spring loaded retractable supply hose mechanisms used for panning out, retraction and the storage of hoses used for oxygen, other gases, treatments or fluids, including intravenous lines, and specifically to this inventor's prior art, the Retractable Oxygen Supply Hose Mechanism, U.S. Pat. No. 6,065,490. In the course of production of the mechanism, some problems in design have been discovered leading to corrections and other improvements being made that are more efficient and cost effective. The problems discovered included but are not limited to binding and kinking of hoses resulting in decreased oxygen flow, slippage of the supply hose reel, slippage of the swivel hose connector, impedance of the free movement of the supply hose and the effectiveness of the spring mechanism design.

When oxygen supply hoses are strung out and cluttering floors they are not only a hazard to all, but also an inconvenience to the patient, therapist and caregivers who must manually roll out, roll up and tie the hose. Further, the continuous exposure of greater lengths of the oxygen supply-hose to ambient temperatures can lead to moisture accumulation in the hose, cannulas and mask. Through a simple tug on the supply hose one controls the amount of length desired. Further, as often as required, the hose and connectors are quickly replaced by opening the “Mechanism Housing Cover”, removing the “Supply-Hose Reel” that includes the supply hose and the swivel connector, and replacing the used “Supply-Hose Reel” with a sterile one. This replaceable “Supply-Hose Reel” has the added feature of A molded in hose connector fitting which is the removable cap now incorporated within the central hub of the upper flange or the upper disc of the reel, eliminating the need for an additional part lowering the cost of the complete replacement unit of the “Supply-Hose Reel” with the new connector and hose. This feature will lower production cost making it more affordable for patients desiring the convenience, for insurers, and also for those patients or caregivers who for any reason are unable to rethread the original reel. For all others, the replacement of the hose and connector is done by opening the “Mechanism Housing Cover”, then panning out all of the hose, removing the reel, removing the “Removable Cap” to gain access to the “Swivel Connector” and the end of the supply hose connected to it, disconnecting the used hose, removing the used connector, inserting a new sterile “Supply-Hose” through the ramp in the barrel of the reel and up through the top of the reel, inserting that end of the hose through the center of the removable cap, inserting the “Swivel Connector” into that end of the “Supply-Hose” that holds it firmly in its position in the removable cap, reinstalling or snapping the “Adaptor Cap” back into its position in the central hub of the “Supply Hose Reel”, repositioning the “Supply-Hose Reel” onto the “Spring Loaded Disc”, then tugging on the hose causing the reel to retract thus wrapping the “Supply-Hose” about the reel and finally closing the “Mechanism Housing cover” ending the process.

### BRIEF SUMMARY OF THE INVENTION

This invention relates to the modifications of and redesigning of spring loaded retractable supply hose mechanisms used for panning out, retraction and the storage of hoses used for oxygen, other gases, treatments or fluids, including intravenous lines, and specifically to this inventor's prior art, the Retractable Oxygen Supply Hose Mechanism, U.S. Pat. No. 6,065,490. Improvements have been made to the “Adaptor Cap” or “Removable Cap” to prevent slippage of the Swivel connector and also include incorporating the “Removable Cap” into the upper disc or flange of the supply-hose reel and/or into the mechanism housing cover that acts as an “anti-rotational” device. A bearing surface is designed into both the reel's upper surface and the interior surface of the mechanism housing cover around the central hubs preventing reel slippage and containing the end play of the reel. This permits the use of four “Post” that extend from the lower disc of the reel into matching holes in the spring loaded disc eliminating the need for a bolt to secure them together which impeded the movement of the supply hose and further preventing reel slippage. A newly designed supply hose reel prevents binding and kinking of the patient's supply hose. A newly designed spring loaded disc and mechanism base together form an internal ratchet system that more effectively facilitates the storage and the quick controlled release of the desired amount of oxygen supply hose for the patient's Medical Oxygen and other gases delivery, therapy, or treatments.

### BRIEF DESCRIPTION OF THE SEVERAL VIEWS OF THE DRAWING

FIG. 1 is a perspective view of the assembled

Retractable Oxygen Supply-Hose Reel with its Housing Cover and Ratchet Base.

FIG. 2 is an exploded frontal view of the Retractable Oxygen Supply-Hose Reel with its Housing Cover and Ratchet Base.

FIG. 3 is an exploded perspective view of the Retractable Oxygen Supply-Hose Reel with its Housing Cover and Ratchet Base.

FIG. 4 is a perspective view of the Mechanism's Housing Cover including the incorporated Anti-rotational cap.

FIG. 5 is a perspective view of the optional Snap-on “Adaptor Cap” or “Removable cap”.

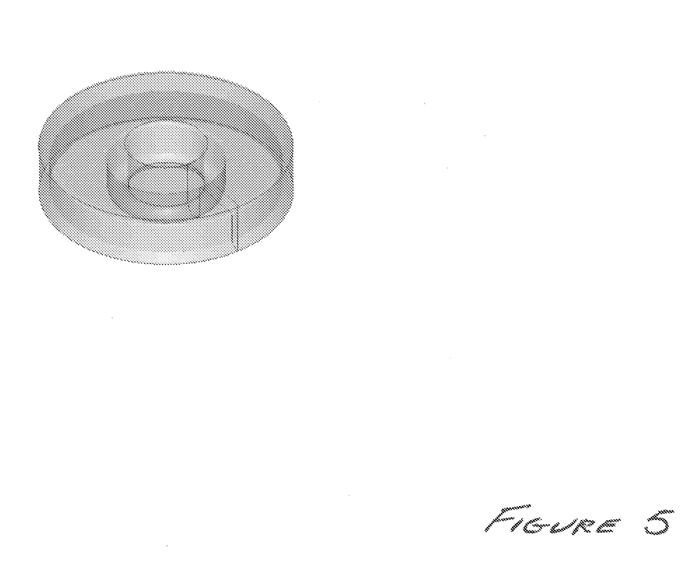

FIG. 6 is a perspective view of the Supply-Hose Reel including the incorporated Cap.

FIG. 7 is a perspective view of the Re-Threadable Supply-Hose Reel without the Removable Cap.

FIG. 8 is a bottom view of the supply hose reel showing the “Paisley” shaped “Soft Curve” at the barrel's base.

FIG. 9 is a view of the Spring Loaded Disc or the “turntable” with the Pawl and Spring and the Ratchet System or teeth and interdental spaces.

### DETAILED DESCRIPTION OF THE INVENTION

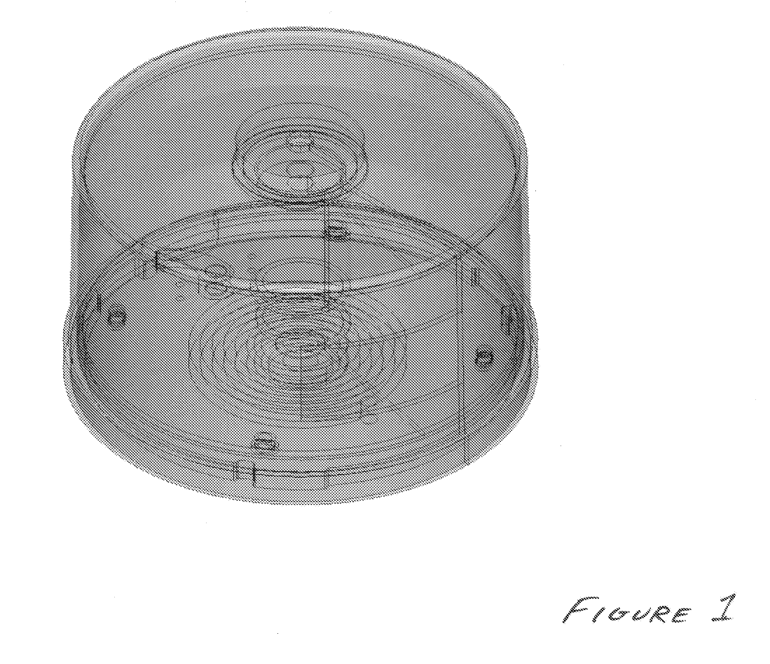

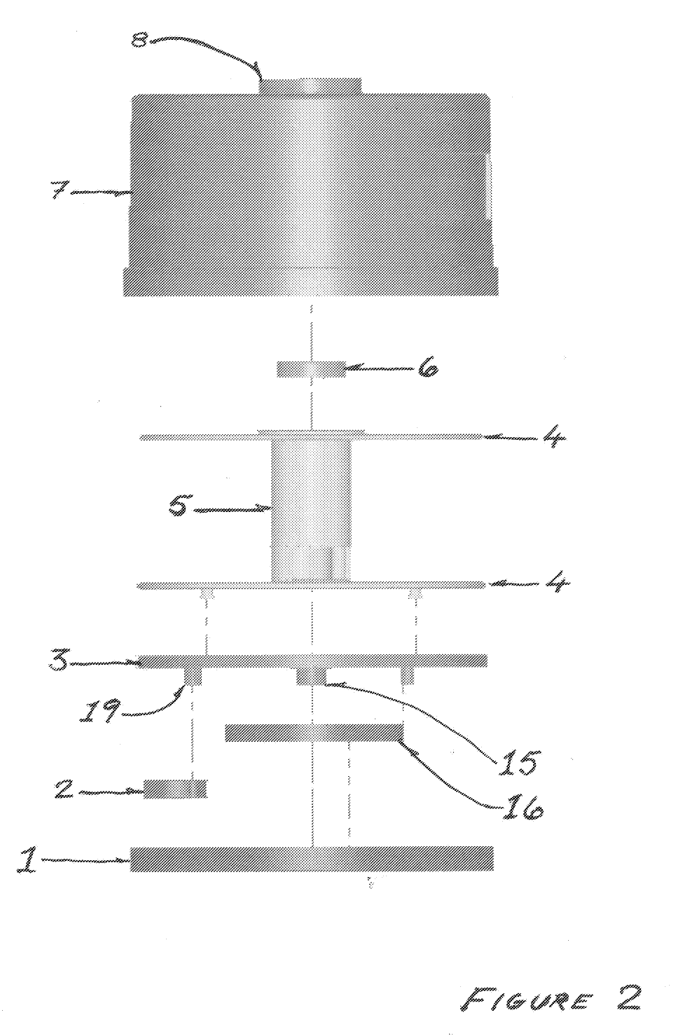

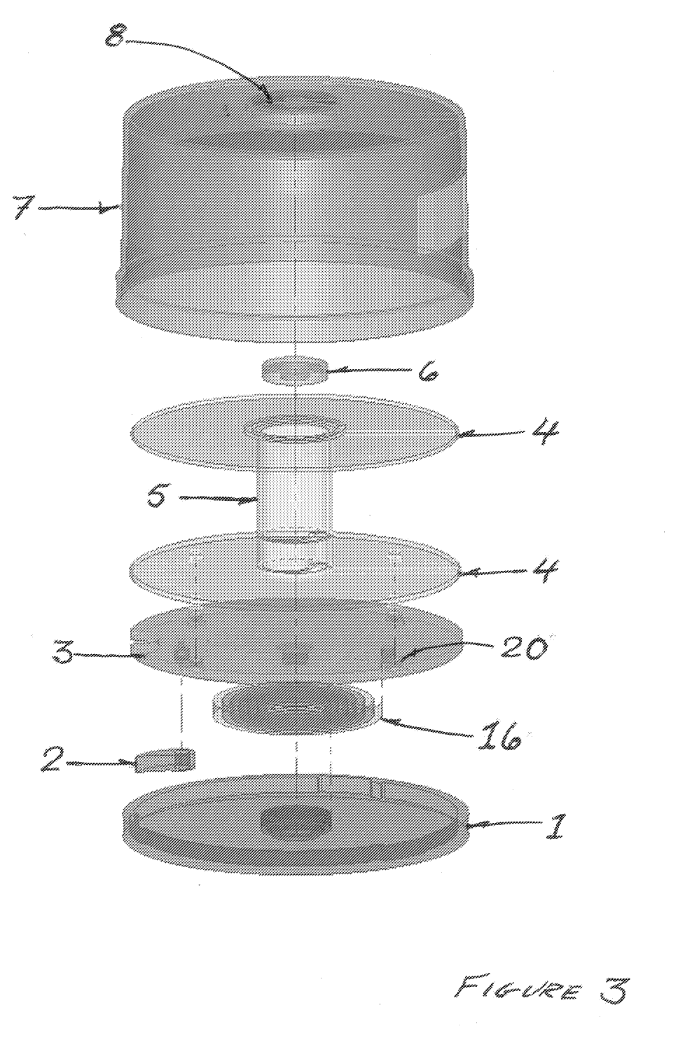

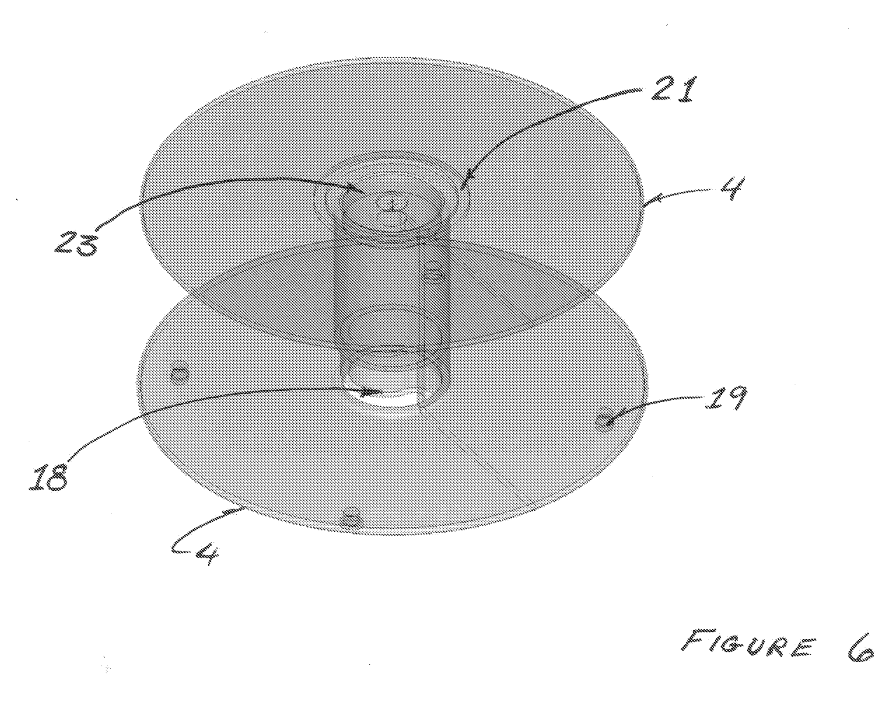

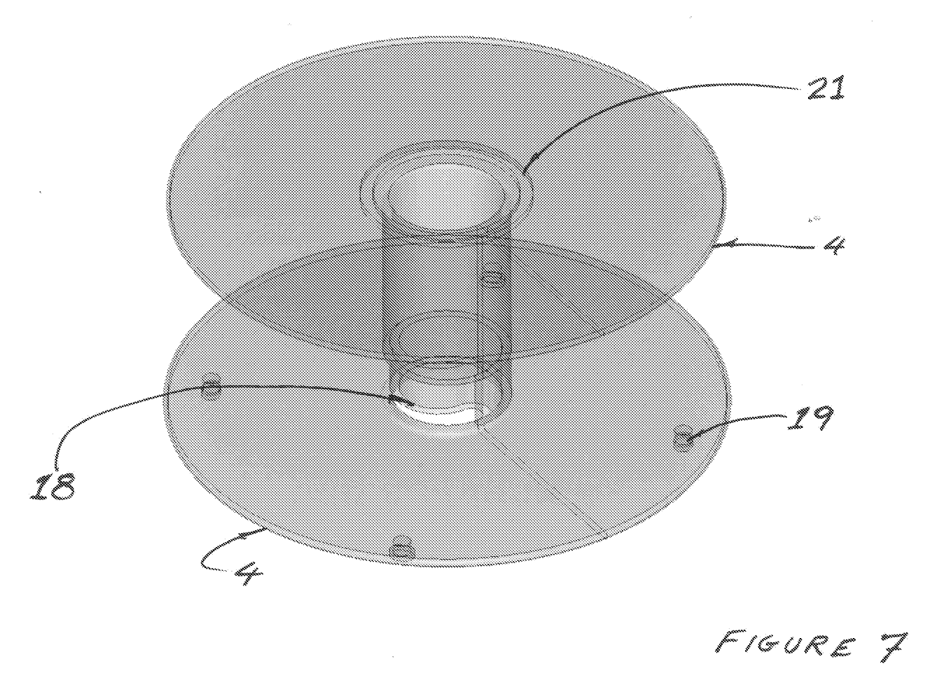

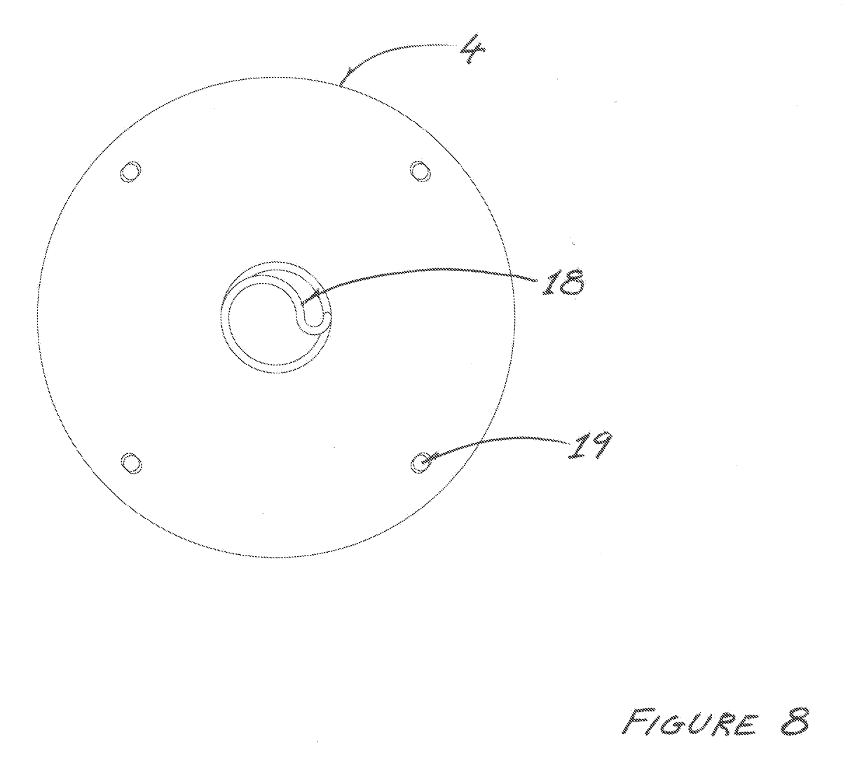

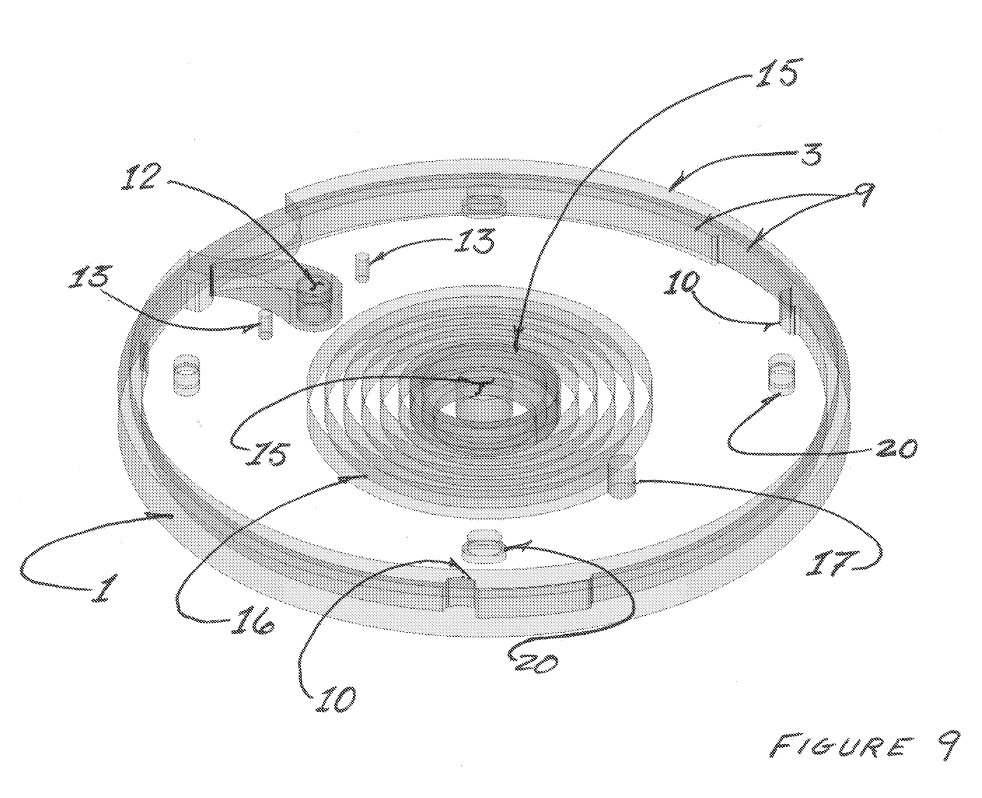

Referring to FIG. 1 and FIG. 2, this invention is designed to modify and redesign spring loaded retractable supply hose mechanisms used for panning out, retraction and the storage of hoses used for oxygen, other gases, treatments or fluids, including intravenous lines, and specifically to this inventor's prior art, the Retractable Oxygen Supply Hose Mechanism, U.S. Pat. No. 6,065,490. The exploded views reveal how the newly designed mechanism fits together to permit improved functioning of all parameters. The parts of the complete mechanism on the exploded frontal view (FIG. 2) and the exploded perspective view (FIG. 3) are from the bottom to the top “The Mechanism Base” at 1, the “Pawl” at 2 which is the “Tear Drop Shaped” part of the ratcheting system, the “Spring-loaded Disc” or “Plate” at 3 that resembles a record player's turntable, the “Supply-Hose Reel” with its “Top and Bottom Plates or Discs or flanges” at 4 connected to the “Hollow Barrel” at 5, the Supply-Hose Reel “Removable Cap” at 6, the “Mechanism Housing” or “Cover” at 7, and the Mechanism Housing “Adaptor Cap” at 8. In FIG. 9, Mechanism Base at 1, is a circular plate that has been molded to form a ratchet on the inside peripheral of this plate that consist of notches or interdental spaces at 9 and teeth at 10 set at an angle for approximately 120 degrees (more or less) of the circumference of the plate. Further, in each interdental space at 10, there is a deeper notch at 11, which allows the pawl to “over-center”, which puts the mechanism into the “Spring Return” mode. That will permit the pawl at 2, a pivoted tongue bolt that is attached to the bottom of the Spring-loaded Disc adapted to fall into the deeper within the interdental spaces on the ratchet part so as to permit motion freely when retracting the patient supply hose and locking it into place when the desired length is released and set into position by a simple tug on the patient supply hose. The pawl at 2 is connected to a “Pawl Post” at 12, that protrudes from the bottom of the Spring-loaded disc 3, said post passes through the center of the circular portion of the pawl which permits the pawl to move from side to side as the spring loaded disc rotates. The pawl is spring loaded to two smaller post items 13 that also protrude from the bottom of the spring loaded disc located on each side of the pawl that allows the angular shaped tongue part of the pawl to shift alternately away and back toward each of the smaller post as the spring loaded disc rotates. The “Central Bearing Housing” of the Mechanism Base at 14 contains a Circular Ball Bearing (not illustrated) press fit or otherwise attached by any other means into the center race of said ball bearing allowing the Spring-Loaded Disc to spin freely in either direction. The Spring-loaded Disc's or Turntables “Central Axle” at 15 protrudes down into the central hub of the Mechanism Base. The flat “Clock Spring” at 16 is then attached on one end to the “Bearing Housing” (not illustrated) and the other end is attached to a more peripherally positioned “Large Spring Post” at 17 also protruding from the bottom of the turntable which is situated at a distance away from the center axle post at 15 which accommodates the length of spring needed to provide the adequate amount of tension to safely release and retract the patient's supply hose. To prevent binding and kinking of the patient supply hose that passes through the outer part of the barrel of the reel through its hollow core up to the swivel connector, a newly designed curved ramp at 18 is created that is “Paisley Shaped” as viewed in FIGS. 6 and 7 and also viewed from the bottom of the reel (FIG. 8). This design allows the patient supply hose to pass softly in a gentle curve into the hollow reel core.

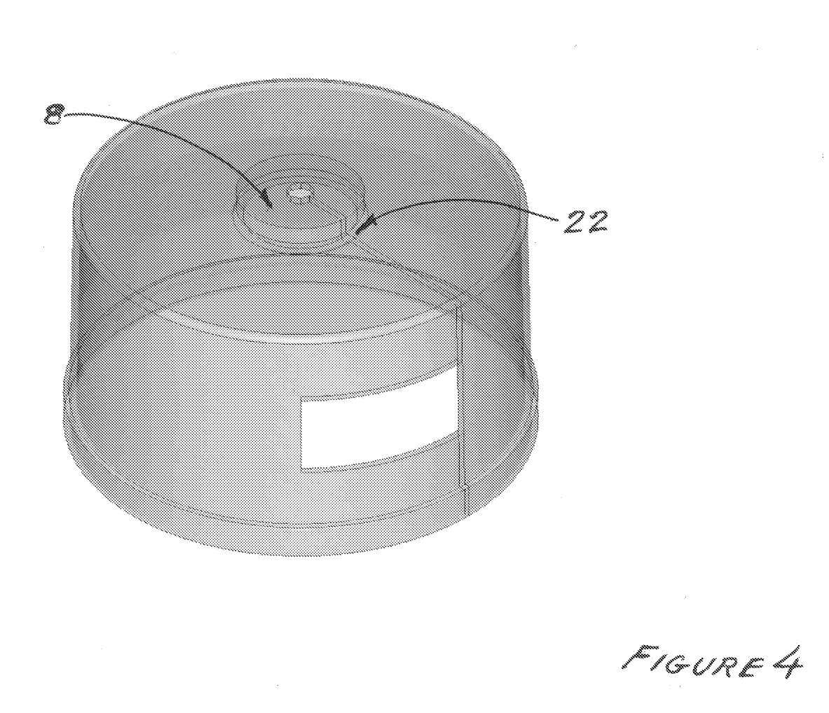

The Supply Hose Reel bottom plate was originally attached with “Single Winged” tabs which were subject to slippage and was secured by a wing bolt to the “Spring Loaded Disc” plate that was subject to impeding hose movement. The Supply Hose Bottom Plate at 4 is now improved and it is held into place by milled “DOUBLE WINGED Posts” at 19 that extend from beneath the Supply-Hose Reel's Bottom Plate and insert into matching width holes in the Spring-loaded disc plate at 20 which lock the reel into place when it moves in both a clockwise and a counterclockwise rotation. The Supply-Hose Reel is also held down by a “bearing surface” at 21 (FIGS. 6 and 7) which further engages a matching “Bearing Surface” at 22 which is situated on the interior surface of the top of the “Mechanism Housing Cover” (FIG. 4). Optionally, the Supply-Hose Reel can also be secured by a recessed “Flat Head Screws” or any other means that does not impede the Supply Hose movement. The “Spring Loaded Disc” is now attached to the Base and forms together a “Single Pawl Internal Toothed Ratchet System” in which the Pawl is moving and the Ratchet is fixed similar to the ratchet system of Franz Reufeaux that is depicted in his book the “Constructor” of 1893. This simple designed Ratcheting System is a modification for use with Oxygen, other gaseous and/or Fluid hose reels.

The Supply-Hose Reel Adaptor cap at 6 that holds the swivel connector not illustrated to the upper surface and the “Central Hub” of the top plate of the Supply Hose Reel at 23 is attached by any known means such as bonding, welding, bolting or snapping it into place into the center hole of the Top Plate of the Supply Hose Reel locking it into the Core of the Supply Hose Reel's Hollow Core or Barrel. The Supply Hose Reel Adaptor Cap can also be incorporated into the “Top Plate or Flange” of the “Supply-Hose Reel” eliminating the need for a separate Adaptor Cap. The newly designed “Mechanism Housing Cover” at 7 permits an anti-rotational Mechanism Housing Adaptor Cap at 8 that allows the lower portion of the swivel connector (not illustrated) to turn freely with the patient Supply Hose Reel and stabilizes the upper portion of the swivel connector (not illustrated) attached to the oxygen source hose that prevents binding and kinking of said hose. As with the Supply Hose Reel Adaptor, the Mechanism Housing Adaptor Cap is attached by any known means such as bonding, welding, bolting or snapping it into place into the center hole of the Top Plate of the Mechanism Housing Cover and locking it into place.

We claim:

1. An Oxygen, gaseous and fluid(s) Supply Hose Mechanism Housing composed of a Supply Hose Mechanism Base and a Supply Hose Mechanism Cover: said Supply Hose Mechanism Base being comprised of a one inch thick circular plate that has been molded to form a ratchet on the inside peripheral of this plate that consist of notches or interdental spaces and Teeth with a “Central Bearing Housing” at its center through which a central axle may pass through and be secured to the base, that houses a “Flat Clock Spring” that attaches on one end to said Central Bearing Housing” and the other end to a more peripherally placed “Large Spring Post” allowing the spring to coil and recoil freely.
The newly designed “Mechanism Housing Cover” permits an anti-rotational Mechanism Housing Adaptor Cap to be connected to the central hub of the top plate of the Mechanism cover that stabilizes the upper portion of a swivel connector to connect to the oxygen source preventing the binding and kinking of the Oxygen Supply hose.
Said Mechanism Housing cover is optionally designed to incorporate the anti-rotational adaptor cap into the top plate of the Mechanism housing cover. The Mechanism Housing Cover has a “bearing surface” located in the interior and central portion of the Housing Cover” that protrudes down to the Supply-Hose Reel's top plate bearing surface.

2. A newly designed Spring Loaded Disc or Plate or “turntable” that has a “teardrop” shaped Pawl connected to a post that protrudes from the bottom of the Spring Loaded Disc that is spring loaded to two small post that also protrude from the bottom of the Spring Loaded Disc that permit the pawl to move back and forth as it passes between the teeth and smooth space of the Mechanism Base, and said Disc having a center Axle that protrudes from the bottom of the Spring Loaded Disc that is attached to the Center Race of the ball bearing housing located in the hub of the said Mechanism Base.
The “Spring Loaded Disc” is now attached to the Base and form an “Internal Toothed Ratchet System” in which the Pawl is moving and the Ratchet is fixed similar to the ratchet system of Franz Reufeaux that is depicted in his book the “Constructor” of 1893. This simple designed Ratcheting System is a modification Reufeaux's ratchet system for use with Oxygen, other gaseous and/or Fluid supply hose reels.

3. A supply hose reel that includes 4 Double winged Post that prevent slippage when the reel moves in a clockwise and counterclockwise rotation.
Said reel having a newly designed “Paisley Shaped” hose Ramp located at the base of the Reel's hollow barrel near the top surface of the bottom plate of the Supply Hose Reel that prevents pinching of the patient's supply hose,
Said reel having a newly designed Supply Hose Reel Adaptor Cap that can either be snapped into the central hub of the top of the Supply Hose Reel or be incorporated into the top plate of the Reel that allows the patient supply hose to pan out and retract without binding the hose.
Said reel having a newly designed “Matching Bearing Surface,” that protrudes upward from around the central hub of the “Adaptor Cap” location that articulates against the “Matching Bearing Surface” that protrudes downward from around the central hub of the “anti-rotational adaptor cap” of the interior surface of the “Mechanism Housing Covering”.
